(19) United States
(12) Patent Application Publication
(10) Pub. No.: US 2009/0076306 A1
(43) Pub. Date: Mar. 19, 2009

(54) PROCESS FOR CONTINUOUS PREPARATION OF A PRIMARY AROMATIC AMINE

(75) Inventors: Frank Haese, Dietzenbach (DE); Joachim Wulff-Doring, Frankenthal (DE); Ulrich Kohler, Mannheim (DE); Peter Gaa, Worms (DE); Frank-Friedrich Pape, Kleinniedesheim (DE); Johann-Peter Melder, Bohl-Iggelheim (DE); Manfred Julius, Limburgerhof (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/159,099

(22) PCT Filed: Dec. 21, 2006
(86) PCT No.: PCT/EP2006/070074

§ 371 Date: Sep. 4, 2008

(30) Foreign Application Priority Data
Jan. 5, 2006 (DE) 102006000995.9

(51) Int. Cl. C07C 209/00; C07C 211/46

(52) U.S. Cl. 564/402; 564/305

(57) ABSTRACT

A process for continuously preparing a primary aromatic amine by reacting a corresponding cycloaliphatic alcohol with ammonia in the presence of hydrogen at a temperature in the range from 80 to 350° C. in the presence of a heterogeneous catalyst, wherein the catalytically active composition of the catalyst, before its reduction with hydrogen, comprises
from 90 to 99.8% by weight of zirconium dioxide (ZrO₂),
from 0.1 to 5.0% by weight of oxygen compounds of palladium and
from 0.1 to 5.0% by weight of oxygen compounds of platinum.

The present invention relates to a process for continuously preparing a primary aromatic amine by reacting a corresponding cycloaliphatic alcohol with ammonia in the presence of hydrogen at a temperature in the range from 80 to 350° C. in the presence of a heterogeneous catalyst.

Aromatic amines are important starting compounds for the preparation of medicaments and crop protection active ingredients. They also find use in the production of plastics. These compounds are therefore of great economic significance and various processes have been developed for their preparation.

A known method is the hydrogenation of aromatic nitro compounds (Houben-Weyl, Methoden der organischen Chemie [Methods of organic chemistry], 4th edition vol. 11/1, page 360 ff.). A disadvantage is that the preceding nitration in the case of substituted aromatics often affords a mixture of several nitro products or regioisomers.

In order to obtain a uniform product, the possibility exists of synthesizing aniline and substituted anilines from the corresponding cyclohexanol and/or cyclohexanone derivatives.

U.S. Pat. No. 3,553,268 (Witco Chemical Comp.) teaches the preparation of aniline from mixtures of cyclohexanol, cyclohexanone and ammonia over a nickel catalyst. A disadvantage here appears to be that this process is not applicable to substituted aromatic amines. In addition, it is restricted in a disadvantageous manner by the fact that apparently only mixtures comprising a maximum of 65% by weight of cyclohexanol can be used.

U.S. Pat. No. 3,442,950 and U.S. Pat. No. 3,347,921 (both Halcon International, Inc.) teach the reaction of mixtures of cyclohexanol and cyclohexanone and ammonia or primary amines over dehydrogenation catalysts, e.g. Pt/C. A disadvantage here is the formation of relatively high-boiling products and other secondary components, such as imines, cyclohexylamine, phenol, diphenylamine and phenylcyclohexylamine, which necessitates a complicated process and workup.

EP 50 229 A1 (BASF AG) describes the reaction of cyclohexanol, cyclohexanol derivatives, cyclohexanone, cyclohexanone derivatives or mixtures thereof in an ammonia/hydrogen atmosphere and over a palladium catalyst. A disadvantage of these processes is that, to achieve good yields, the catalyst must comprise not only palladium but also zinc and the very poisonous cadmium. When zinc and cadmium are omitted, the yields worsen considerably. When the cadmium is omitted from the catalyst, it is apparently necessary to add various aliphatic amines such as N-methylmorpholine or N-methylpiperidine for a good yield. In further examples, it is possible to synthesize the corresponding aromatic amines in good yields even without cadmium and even without added aliphatic amine over the catalyst. However, this is possible only because either the amount of palladium in the catalyst has been increased drastically or the shaped bodies have been decreased greatly in size. A disadvantage of this is that a high noble metal content makes the catalyst very expensive, and excessively small shaped bodies unfavorably increase the back pressure and are problematic to handle.

It was thus an object of the present invention to overcome one or more disadvantages of the prior art by discovering an improved, economically viable process for preparing a primary aromatic amine. It was a particular aim to find a process by which aniline and appropriately substituted aromatic amines are preparable in good yields from cyclohexanol and substituted cyclohexanols, without having to accept the disadvantages of the prior art, in particular the disadvantages described above.

Accordingly, we have found a process for continuously preparing a primary aromatic amine by reacting a corresponding cycloaliphatic alcohol with ammonia in the presence of hydrogen at a temperature in the range from 80 to 350° C. in the presence of a heterogeneous catalyst, wherein the catalytically active composition of the catalyst, before its reduction with hydrogen, comprises

from 90 to 99.8% by weight of zirconium dioxide (ZrO₂),
from 0.1 to 5.0% by weight of oxygen compounds of palladium and
from 0.1 to 5.0% by weight of oxygen compounds of platinum.

According to the invention, aniline and substituted aromatic amines, especially substituted anilines, can be prepared without the disadvantages described when cyclohexanol or correspondingly substituted cyclohexanol is reacted with ammonia and hydrogen at elevated temperature over the bimetallic zirconium dioxide catalyst which comprises platinum and palladium.

For the synthesis in the gas phase, the reactant alcohol is evaporated in a controlled manner, preferably in a cycle gas stream, and fed in gaseous form to the reactor. The cycle gas serves firstly to evaporate the reactant alcohol and secondly as a reactant for the amination.

In the cycle gas method, the starting materials (alcohol, hydrogen and ammonia) are evaporated in a cycle gas stream and fed in gaseous form to the reactor.

The reactants (alcohol and ammonia) can also be evaporated as aqueous solutions and conducted to the catalyst bed with the cycle gas stream.

Preferred reactors are tubular reactors. Examples of suitable reactors with cycle gas stream can be found in Ullmann's Encyclopedia of Industrial Chemistry, 5th Ed., Vol. B 4, pages 199-238, “Fixed-Bed Reactors”.

Alternatively, the reaction is advantageously effected in a tube bundle reactor or in a single-stream plant.

In a single-stream plant, the tubular reactor in which the reaction proceeds can consist of a series connection of a plurality of (e.g. two or three) individual tubular reactors. Optionally, an intermediate introduction of feed (comprising the reactant and/or ammonia and/or H₂) and/or cycle gas and/or reactor effluent from a downstream reactor is possible here in an advantageous manner.

There is preferably no series connection of two or more reactors.

The cycle gas flow rate is preferably in the range from 40 to 1500 m³ (at operating pressure)/[m³ of catalyst (bed volume)·h], in particular in the range from 100 to 700 m³ (at operating pressure)/[m³ of catalyst (bed volume)·h].

The cycle gas comprises preferably at least 10% by volume, particularly from 50 to 100% by volume, very particularly from 80 to 100% by volume of H₂.

For the synthesis in the liquid phase, suitable reactants and products are all of those which have high boiling points or are thermally labile. In these cases, a further advantage is that it is possible to dispense with evaporation and recondensation of the amine in the process.

In the process according to the invention, the catalysts are preferably used in the form of catalysts which consist only of catalytically active composition and, if appropriate, a shaping assistant (for example graphite or stearic acid) if the catalyst is used as a shaped body, i.e. do not comprise any further catalytically active ingredients. In this connection, the oxidic support material zirconium dioxide (ZrO₂) is considered to be included in the catalytically active composition.

The catalysts are used in such a way that the catalytically active composition ground to powder is introduced into the reaction vessel or that the catalytically active composition, after grinding, mixing with shaping assistants, shaping and heat treatment, is arranged in the reactor as shaped catalyst bodies—for example as tablets, spheres, rings, extrudates (e.g. strands).

The concentration data (in % by weight) of the components of the catalyst are based in each case, unless stated otherwise, on the catalytically active composition of the finished catalyst after its last heat treatment and before its reduction with hydrogen.

The catalytically active composition of the catalyst, after its last heat treatment and before its reduction with hydrogen, is defined as the sum of the masses of the catalytically active constituents and of the abovementioned catalyst support materials, and comprises essentially the following constituents:

zirconium dioxide (ZrO₂), oxygen compounds of palladium and oxygen compounds of platinum.

The sum of the abovementioned constituents of the catalytically active composition is typically from 70 to 100% by weight, preferably from 80 to 100% by weight, more preferably from 90 to 100% by weight, particularly >95% by weight, very particularly >98% by weight, in particular >99% by weight, for example more preferably 100% by weight.

The catalytically active composition of the catalysts used in the process according to the invention may also comprise one or more elements (oxidation stage 0) or their inorganic or organic compounds selected from groups I A to VI A and I B to VII B and VIII of the Periodic Table of the Elements.

Examples of such elements and their compounds are:

transition metals such as Co or CoO, Re or rhenium oxides, Mn or MnO₂, Mo or molybdenum oxides, W or tungsten oxides, Ta or tantalum oxides, Nb or niobium oxides or niobium oxalate, V or vanadium oxides or vanadyl pyrophosphate; lanthanides such as Ce or CeO₂, or Pr or Pr₂O₃; alkali metal oxides such as Na₂O; alkali metal carbonates; alkaline earth metal oxides such as SrO; alkaline earth metal carbonates such as MgCO₃, CaCO₃ and BaCO₃; boron oxide (B₂O₃).

The catalytically active composition of the catalysts used in the process according to the invention preferably comprises no ruthenium, no copper, no cadmium, no zinc, no cobalt, no iron and/or no nickel.

The catalytically active composition of the catalysts used in the process according to the invention, after its last heat treatment and before its reduction with hydrogen, comprises

from 90 to 99.8% by weight, preferably from 98 to 99.6% by weight, more preferably from 98.8 to 99.2% by weight of zirconium dioxide (ZrO₂),
from 0.1 to 5.0% by weight, preferably from 0.2 to 1.0% by weight, more preferably from 0.4 to 0.6% by weight of oxygen compounds of palladium and
from 0.1 to 5.0% by weight, preferably from 0.2 to 1.0% by weight, more preferably from 0.4 to 0.6% by weight of oxygen compounds of platinum.

For the preparation of the catalysts used in the process according to the invention, various processes are possible. Mention should be made here, for example, of the known precipitation methods.

The catalysts used in the process according to the invention may be prepared in particular by impregnating zirconium dioxide (ZrO₂) which is present, for example, in the form of powder or shaped bodies such as extrudates, tablets, spheres or rings.

The zirconium dioxide is used, for example, in the monoclinic or tetragonal form, preferably in the monoclinic form.

Shaped bodies can be produced by the customary processes.

The impregnation is likewise effected by the customary processes, as described, for example, in A. B. Stiles, Catalyst Manufacture—Laboratory and Commercial Preparations, Marcel Dekker, New York (1983), by applying an appropriate metal salt solution in each case in one or more impregnation stages, the metal salts used being, for example, appropriate nitrates, acetates or chlorides. After the impregnation, the composition is dried and optionally calcined.

The impregnation can be effected by the so-called incipient wetness method, in which the zirconium dioxide is moistened in accordance with its water uptake capacity, up to a maximum of saturation with the impregnation solution. The impregnation can also be effected in supernatant solution.

In the case of multistage impregnation processes, it is appropriate to dry and optionally to calcine between individual impregnation steps. Multistage impregnation can be employed particularly advantageously when the zirconium dioxide is to be loaded with a relatively large amount of metal.

To apply the metal components to the zirconium dioxide, the impregnation can be effected simultaneously with all metal salts or successively in any sequence of the individual metal salts.

After the calcination, which is carried out, for example, at a temperature in the range from 200° C. to 600° C., the catalyst is appropriately conditioned, whether it be by grinding to a certain particle size or by mixing it, after it has been ground, with shaping assistants such as graphite or stearic acid, compressing it by means of a press to moldings, for example tablets, and heat-treating. The heat treatment temperatures correspond preferably to the temperatures in the calcining.

The catalysts prepared in this way comprise the catalytically active metals in the form of a mixture of their oxygen compounds, in particular in the form of oxides and mixed oxides.

The catalysts prepared in this way are stored as such and, if appropriate, treated. Before they are used as catalysts, they are typically prereduced. However, they can also be used without prereduction, in which case they are reduced under the conditions of the hydrogenating/dehydrogenating amination by the hydrogen present in the reactor.

For prereduction, the catalysts are exposed to a nitrogen-hydrogen atmosphere first at preferably from 150 to 200° C. over a period of, for example, from 12 to 20 hours, and then treated in a hydrogen atmosphere at preferably from 200 to 400° C. for another up to approx. 24 hours. In this prereduction, a portion of the oxygen-containing metal compounds present in the catalysts is reduced to the corresponding metals, so that they are present together with the different types of oxygen compounds in the active form of the catalyst.

Selected Pd/Pt/ZrO₂ catalysts disclosed in EP-A-701 995 (BASF AG) are used with particular preference in the process according to the invention.

The process according to the invention is carried out continuously, the catalyst preferably being arranged as a fixed bed in the reactor. The flow to the fixed catalyst bed may either be from above or from below. The gas stream is adjusted by temperature, pressure and amount so that even relatively high-boiling reaction products remain in the gas phase.

The aminating agent, ammonia, can be used in stoichiometric, substoichiometric or superstoichiometric amounts based on the alcoholic hydroxyl group to be aminated.

Ammonia is used generally with a 1.5 to 250-fold, preferably 2 to 100-fold, in particular 2 to 10-fold molar excess per mole of alcoholic hydroxyl group to be converted.

Higher excesses of ammonia are possible.

Preference is given to employing an offgas flow rate of from 5 to 800 standard cubic meters/h, in particular from 20 to 300 standard cubic meters/h.

When working in the liquid phase, the reactants (alcohol plus ammonia) are passed simultaneously, including hydrogen, over the catalyst, which is typically disposed in a fixed bed reactor preferably heated externally, in the liquid phase at pressures of generally from 5 to 30 MPa (50-300 bar), preferably from 5 to 25 MPa, more preferably from 15 to 25 MPa, and temperatures of generally from 80 to 350° C., particularly from 100 to 300° C., preferably from 120 to 270° C., more preferably from 130 to 250° C., in particular from 170 to 230° C. Both a trickle mode and a liquid-phase mode are possible. The catalyst hourly space velocity is generally in the range from 0.05 to 5 kg, preferably from 0.1 to 2 kg and more preferably from 0.2 to 0.6 kg of alcohol per liter of catalyst (bed volume) and hour. If appropriate, the reactants can be diluted with a suitable solvent such as tetrahydrofuran, dioxane, N-methylpyrrolidone or ethylene glycol dimethyl ether. It is appropriate to heat the reactants before they are fed into the reaction vessel, preferably to the reaction temperature.

When working in the gas phase, the gaseous reactants (alcohol plus ammonia) are passed over the catalyst in the presence of hydrogen in a gas stream, preferably hydrogen, selected so as to be sufficiently large for evaporation, at pressures of generally from 0.1 to 40 MPa (from 1 to 400 bar), preferably from 0.1 to 10 MPa, more preferably from 0.1 to 5 MPa. The temperatures for the amination are generally from 80 to 350° C., particularly from 100 to 300° C., preferably from 120 to 290° C., more preferably from 160 to 280° C. The flow to the fixed catalyst bed may be either from above or from below. The required gas stream is preferably obtained by a cycle gas method.

The catalyst hourly space velocity is generally in the range from 0.01 to 2 and preferably from 0.05 to 0.5 kg of alcohol per liter of catalyst (bed volume) and hour.

The hydrogen is fed to the reaction generally in an amount of from 5 to 400 I, preferably in an amount of from 50 to 200 I per mole of alcohol component, the amounts in liters each having been converted to standard conditions (S.T.P.).

Both when working in the liquid phase and when working in the gas phase, it is possible to use higher temperatures and higher overall pressures and catalyst hourly space velocities. The pressure in the reaction vessel, which results from the sum of the partial pressures of the aminating agent ammonia, of the alcohol and of the reaction products formed and, if appropriate, of the solvent used at the temperatures specified, is appropriately increased by injecting hydrogen up to the desired reaction pressure.

Both in the case of continuous operation in the liquid phase and in the case of continuous operation in the gas phase, the excess aminating agent can be circulated together with the hydrogen.

When the catalyst is arranged as a fixed bed, it may be advantageous for the selectivity of the reaction to mix the shaped catalyst bodies in the reactor with inert packings, to “dilute” them as it were. The proportion of packings in such catalyst preparations may be from 20 to 80 parts by volume, particularly from 30 to 60 parts by volume and in particular from 40 to 50 parts by volume.

The water of reaction formed in the course of the reaction (in each case one mole per mole of alcohol group converted) generally does not have a disruptive effect on the degree of conversion, the reaction rate, the selectivity and the catalyst lifetime, and is therefore appropriately not removed therefrom until the workup of the reaction product, for example by distillation.

After the reaction effluent has appropriately been decompressed, the excess hydrogen and any excess aminating agent present are removed therefrom and the resulting crude reaction product is purified, for example by a fractional rectification. The excess aminating agent and the hydrogen are advantageously returned back into the reaction zone. The same applies to any incompletely converted alcohol component.

The particular pure products can be obtained from the crude materials by rectification by the known methods. The pure products are obtained as azeotropes with water or can be dewatered by a liquid-liquid extraction with concentrated sodium hydroxide solution according to the patent applications EP-A-1 312 599 und EP-A-1 312 600 (both BASF AG). This dewatering can be effected before or after the purifying distillation. Distillative dewatering in the presence of an azeotroping agent by known methods is also possible.

In the case that the crude material or the aromatic amine in the crude material is barely water-miscible, if at all, dewatering is also possible by a separation of the organic and of the aqueous phase by known methods. According to the procedure taught in EP-A-1 312 599 and in EP-A-1 312 600 (both BASF AG), one or more low boiler fractions of the amine-containing mixture can be removed by distillation in one step from the separated organic phase. In a further step, the possibility exists of removing one or more high boiler fractions from the amine-containing mixture by distillation. In a distillation step which follows, the substantially anhydrous amine can be obtained in pure form from the mixture as a bottom draw or side draw of the column, and is, if appropriate, subjected to a further purification or separation.

These individual steps for purifying the amine can, if appropriate, also be carried out batchwise or continuously in a single column, in which case the low boilers can be removed via the top draw and/or the side draw of the rectifying section of the column, the high boiler fractions can be removed via the bottom draw of the distillation column and the pure amine can be removed via the side draw in the stripping section of the column.

In a particularly preferred variant, the continuous distillation column used is a dividing wall column.

Unconverted reactants and any suitable by-products which are obtained can be returned back into the synthesis. Unconverted reactants can be flowed again in the cycle gas stream over the catalyst bed in batchwise or continuous mode after condensation of the products in the separator.

As the result of the use of ammonia as the aminating agent, the alcoholic hydroxyl group of the cycloaliphatic alcohol used is converted to the primary amino group (—NH₂) while retaining the position on the aliphatic ring.

Suitable cycloaliphatic alcohols are virtually all alcohols having an aliphatic OH function. In other words, the OH group is bonded to an sp³-hybridized carbon atom in an aliphatic ring. In addition to carbon atoms, the aliphatic ring may also have one or more heteroatoms such as N, O or S. The alcohols may also bear substituents or comprise functional groups which behave inertly under the conditions of the hydrogenating/dehydrogenating amination, for example alkoxy, alkenyloxy, alkylamino or dialkylamino groups, or else, if appropriate, are hydrogenated under the conditions of the hydrogenating amination, for example CC double or triple bonds. When polyhydric cycloaliphatic alcohols are to be aminated, it is possible via the control of the reaction conditions to preferentially obtain corresponding amino alcohols or polyaminated products.

For example, the following cycloaliphatic alcohols are preferably aminated: cyclohexanol, where the cyclohexyl radical may bear one or more alkyl radicals, in particular C₁₋₉-alkyl radicals and C₅₋₆-cycloalkyl radicals, and/or aryl radicals as substituents.

C₁₋₉-Alkyl radicals, preferably C₁₋₃-alkyl radicals, are, for example, methyl, ethyl, n-propyl, isopropyl, n-butyl, isobutyl, sec-butyl, tert-butyl, n-pentyl, isopentyl, sec-pentyl, neopentyl, 1,2-dimethylpropyl, n-hexyl, isohexyl, sec-hexyl, cyclopentylmethyl, n-heptyl, isoheptyl, cyclohexylmethyl, n-octyl, isooctyl, 2-ethylhexyl, n-nonyl.

Aryl radicals are, for example, phenyl, 1-naphthyl, 2-naphthyl.

Further examples of cycloaliphatic alcohols used with preference are: 2,6-dimethylcyclohexanol, 2,4-dimethylcyclohexanol, 3,5-dimethylcyclohexanol, 2,3,6-tri-methylcyclohexanol, 2,4,6-trimethylcyclohexanol, 2,6-diethylcyclohexanol, 2-methyl-6-ethylcyclohexanol, 2,6-diisopropylcyclohexanol, 2,6-di-sec-butylcyclohexanol, 3-tert-butylcyclohexanol, 2-isopropyl-6-methylcyclohexanol and 2-isopropyl-6-ethylcyclo-hexanol.

The cycloaliphatic alcohols used as the reactant, especially cyclohexanols, are readily available compounds.

The cycloaliphatic alcohols used as the reactant, especially cyclohexanols, very particularly cyclohexanol and 2,6-dimethylcyclohexanol, preferably have a purity of ≧95% by weight, particularly ≧98% by weight.

The content of ketones, especially of cyclohexanones, is preferably ≦2% by weight, particularly ≦1% by weight and is very particularly in the range from 0 to ≦0.5% by weight, further particularly in the range from 0 to ≦0.2% by weight.

For example, the reactant used may be commercially available 2,6-dimethylcyclohexanol which has a content of at least 70% by weight (for example from Aldrich, ABCR, ASDI-Inter, ICN-RF, VWR), preferably at least 95% by weight (for example from TCI-JP, TCI-US), more preferably at least 99% by weight. The 2,6-dimethylcyclohexanol reactant used with particular preference and having ≧99% by weight purity is offered for sale by various suppliers, for example Acros and Kanto. 98% by weight material is obtained, for example, from Pfaltz-Bauer or Wiley and other suppliers.

Aromatic amines prepared with preference by the process according to the invention, especially substituted anilines, are 2,6-di(C₁₋₉-alkyl)anilines from the corresponding 2,6-di(C₁₋₉-alkyl)cyclohexanols. Examples are 2,6-dimethylaniline (2,6-xylidine), 3,5-dimethylaniline, 2,6-diethylaniline, 2-methyl-6-ethylaniline, 2,6-diisopropylaniline, 2-isopropyl-6-methylaniline and 2-isopropyl-6-ethylaniline.

The aromatic amine prepared with particular preference by the process according to the invention is 2,6-dimethylaniline (2,6-xylidine) by reaction of 2,6-dimethyl-cyclohexanol.

It is possible by the process according to the invention, especially according to claims 17 or 18, in particular to prepare 2,6-dimethylaniline (2,6-xylidine) having a purity of ≧99% by weight, particularly ≧99.5% by weight, very particularly ≧99.85% by weight, and a content of 2,6-dimethylphenol of ≦0.1% by weight, particularly ≦0.05% by weight, very particularly ≦0.02% by weight, for example from 0 to 0.015% by weight, from 2,6-dimethylcyclohexanol and ammonia.

The abovementioned contents in % by weight are determined by gas chromatography as follows:

| | |
|---|---|
| Separating column: | DB WAX (polyethylene |
| | glycol) |
| Length (m): | 30 |
| Film thickness (μm) | 0.5 |
| Internal diameter (mm) | 0.25 |
| Carrier gas: | helium |
| Initial pressure (bar) | 1.0 |
| Split (ml/min.): | 100 |
| Septum purge (ml/min.) | 4 |
| Oven temperature (° C.): | 80 |
| Preheating time (min.) | 3 |
| Rate (° C./min.) | 5 |
| Oven temperature (° C.) | 240 |
| Continued heating time (min.): | 30 |
| Injector temperature (° C.) | 250 |
| Detector temperature (° C.) | 260 |
| Injection | HP 7673 Autosampler |
| Injection volume (microl) | 0.2 |
| Detector type: | FID |

### EXAMPLES

For all examples, the selected bimetallic palladium/platinum catalyst according to Example 4 (page 6, lines 12-15) of EP-B1-701 995 was used and also activated by the method described there (page 4, lines 47-52). Thereafter, the supported noble metal catalyst was installed into the reactor and subsequently reduced at 200° C. in a nitrogen/hydrogen stream at ambient pressure or under operating pressure.

### Preparation of 2,6-dimethylaniline

In a tubular reactor with 10 liter catalyst charge, a cycle gas stream of 170 kg/h of ammonia and 20 kg/h of hydrogen was established at total pressure 2 bar. 122 kg/h of 2,6-dimethylcyclohexanol were added continuously to this stream and evaporated. The gaseous mixture was flowed over the catalyst bed at from 200 to 270° C. The yield of 2,6-xylidine downstream of the reactor was greater than 90%.

1. A process for continuously preparing a primary aromatic amine by reacting a corresponding cycloaliphatic alcohol with ammonia in the presence of hydrogen at a temperature in the range from 80 to 350° C. in the presence of a heterogeneous catalyst, wherein the catalytically active composition of the catalyst, before its reduction with hydrogen, comprises
from 90 to 99.8% by weight of zirconium dioxide (ZrO₂),
from 0.1 to 5.0% by weight of oxygen compounds of palladium and
from 0.1 to 5.0% by weight of oxygen compounds of platinum.

2. The process according to claim 1, wherein the reaction is carried out at a temperature in the range from 120 to 300° C.

3. The process according to claim 1, wherein the reaction is carried out in the liquid phase at an absolute pressure in the range from 5 to 30 MPa or in the gas phase at an absolute pressure in the range from 0.1 to 40 MPa.

4. The process according to claim 1, wherein the catalytically active composition of the catalyst, before its reduction with hydrogen, comprises
from 98 to 99.6% by weight of zirconium dioxide (ZrO₂),
from 0.2 to 1.0% by weight of oxygen compounds of palladium and
from 0.2 to 1.0% by weight of oxygen compounds of platinum.

5. The process according to claim 1, wherein the catalytically active composition of the catalyst, before its reduction with hydrogen, comprises
from 98.8 to 99.2% by weight of zirconium dioxide (ZrO₂),
from 0.4 to 0.6% by weight of oxygen compounds of palladium and
from 0.4 to 0.6% by weight of oxygen compounds of platinum.

6. The process according to claim 1, wherein the ammonia is used in from 1.5 to 250 times the molar amount of the cycloaliphatic alcohol used.

7. The process according to claim 1, wherein the ammonia is used in from 2.0 to 10 times the molar amount of the cycloaliphatic alcohol used.

8. The process according to claim 1, wherein the catalyst is arranged in the reactor as a fixed bed.

9. The process according to claim 1, wherein the reaction is effected in a tubular reactor.

10. The process according to claim 1, wherein the reaction is effected in a cycle gas method.

11. The process according to claim 1, wherein the alcohol is used as an aqueous solution.

12. The process according to claim 1, wherein the ammonia is used as an aqueous solution.

13. The process according to claim 1 for preparing a phenylamine, where the phenyl radical may bear one or more C₁₋₉-alkyl radicals as substituents.

14. The process according to claim 1 for preparing aniline by reacting cyclohexanol with ammonia.

15. The process according to claim 1 for preparing a 2,6-di(C₁₋₉-alkyl)aniline.

16. The process according to claim 1 for preparing 2,6-dimethylaniline (2,6-xylidine) by reacting 2,6-dimethylcyclohexanol with ammonia.

17. The process according to claim 1, wherein the organic phase is removed from the crude reaction product and is subsequently separated by distillation continuously in a distillation column, the primary aromatic amine being drawn off via a side draw in the stripping section of the column, low boilers and water via the top, and high boilers via the bottom.

18. The process according to claim 17, wherein the distillation column is a dividing wall column.

19. 2,6-Dimethylaniline (2,6-xylidine) having a purity of ≧99% by weight and a content of 2,6-dimethylphenol of ≦0.1% by weight, preparable by a process according to claim 17.

20. 2,6-Dimethylaniline (2,6-xylidine) according to claim 19 having a purity of ≧99.5% by weight and a content of 2,6-dimethylphenol of ≦0.05% by weight.
